(19) United States
(12) Patent Application Publication
(10) Pub. No.: US 2010/0062961 A1
(43) Pub. Date: Mar. 11, 2010

(54) Good Foaming Creamy or Paste-Like Cleansers Comprising Floor Levels of Long Chain Lipids or Lipid Mimics

(75) Inventors: Albert Joseph Post, Orange, CT (US); Georgia Shafer, Southbury, CT (US); Kavssery Ananthapadmanabhan, Woodbury, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/205,104

(22) Filed: Sep. 5, 2008

(51) Int. Cl. A61K 8/92; A61Q 19/10

(52) U.S. Cl. 510/158

(57) ABSTRACT

The present invention relates to compositions, especially pastes/creams which have high level of long chain lipids or lipid mimics. These help provide “creaminess”, a desirable rheological characteristic. This is seen from defined viscosity values which define the pastes. In addition, unpredictably, when used in combination with short chain phase inducers, foam values are adequate when it would be expected the lipids would decrease foam. The composition also retains stability from physical separation.

### FIELD OF THE INVENTION

The present invention relates to cleanser compositions, preferably those which are lamellar structured and have cream or paste-like rheology. The compositions comprise at least 2% to 40%, preferably 5% to 30% by wt. anionic surfactant(s) which are selected by having Krafft point (minimum temperature at which surfactant micelles form) low enough to exclude or minimize acyl isethionate as said primary anionic (i.e., small amounts, up to about 5% by wt. acyl isethionate, may be included as co-surfactant). By using critical minimal amounts (floor levels) of long chain lipid or lipid mimics relative to surfactant, compositions of the invention unexpectedly obtain desirable rheology defined by paste-like viscosity (magnitude of dynamic viscosity >50 Pa·s, preferably >100 Pa·s, measured at 25° C. at 1 sec⁻¹ by the procedure described herein) and/or for desirable aesthetics (e.g., stability) while unpredictably retaining good foaming.

### BACKGROUND

Cleansers comprising fatty acids and fatty alcohols generally are not new. See U.S. Pat. No. 5,308,525 to Dias et al.; U.S. Pat. No. 5,234,619 or U.S. Pat. No. 5,132,037, both to Greene et al.

The Greene et al. patents and several others filed by applicants (U.S. Ser. No. 11/613,617; U.S. Ser. No. 11/613,696; U.S. Ser. No. 11/613,666, all to Tsaur et al., filed Dec. 20, 2006) disclose fatty acid containing formulations comprising predominantly acyl isethionate in the surfactant system. Such surfactant has higher Krafft point than that of predominant anionic of the subject invention, meaning the acyl isethionate will more readily remain in a crystalline or solid precipitate phase rather than form micelles at room temperature.

Other references disclose emollients in combination with broader surfactant systems (U.S. Pat. No. 5,994,280 to Giret; U.S. Pat. No. 5,965,500 to Puvvada et al.; WO 2005/107691, assigned to Kimberly). While a number of these references disclose use of fatty acids and/or fatty alcohols (e.g., to help form lamellar phase), none of the references disclose or appreciate the use of critical amounts (weight ratio of sparingly-soluble lipid or lipid mimic to total surfactant plus co-surfactant being greater than 1:2) of long chain lipid mimics, specifically long-chain (>C₁₆) fatty acid and/or alcohols to provide a suitable rheological profile for a creamy or paste-like cleanser, yet retain good foaming and stability. The use of larger amounts of these long chain molecules would have been expected to be strongly de-foaming. These references don't appreciate that inclusion of critical amounts of lipid or mimic provides good skin moisturization (consistent with the desired rheology) while as noted, quite unpredictably providing good foam and stability.

U.S. Pat. No. 5,994,280 to Giret, for example, discloses compositions comprising 5 to 50% mixture of anionic and amphoteric surfactant, 3 to 40% of insoluble nonionic oil and/or wax, 0.1 to 8% by wt. of C₁₀ to C₁₈ weight average chain length fatty acid, citric acid and water. No more than 4% by wt. fatty acid is used in the examples (Example I). There is no recognition of using critical amounts of long chain fatty acid, i.e. having chain length greater than C₁₆, relative to surfactant.

U.S. Pat. No. 5,965,500 to Puvvada discloses use of surfactant, emollient and 0 to 10% by weight of C₂ to C₂₄ fatty acids. Again, there is no recognition that critical amounts (>50% relative to total surfactant) of C₁₆ or higher fatty acid provide excellent aesthetic and rheological benefits (e.g., foaming, creaminess, stability, mildness).

WO 2005/107691 discloses surfactant, lipid phase and water wherein part of lipid phase may comprise stearic acid. There is no disclosure as to what amount of fatty acid is used.

U.S. Pat. No. 5,296,157 to MacGlip et al. discloses compositions comprising 5% to 20% by weight of potassium fatty acid soap and 2.5% to 18% free fatty acid with chain length from C₈ to C₂₂. There is no recognition of the criticality of using critical amounts of long chain fatty acid, and in addition, the viscosity of composition would appear to be well below those of the subject compositions.

### BRIEF DESCRIPTION OF THE INVENTION

Unexpectedly, applicants have now found that, when critical amounts of skin lipid or lipid mimic (e.g., long chain fatty acid, such as C₁₆ or higher straight chain fatty acid and/or fatty alcohol) are used, a cleanser is obtained with enhanced aesthetic and rheological properties. These properties include good foamability, creaminess, mildness, and stability. It is particularly unpredictable to use these levels of relatively insoluble skin lipid or mimic (such as long chain length fatty acids) to provide desirable rheological properties and/or aesthetic properties (e.g., physical stability) while retaining good foaming.

More specifically, the invention comprises a liquid cleanser composition comprising:
(1) 2% to 40%, preferably 5% to 30%, even more preferably 7 to 25% by wt. of an anionic surfactant having a Krafft point below 20° C.;
(2) a co-surfactant selected form the group consisting of anionic, nonionic, amphoteric, zwitterionic surfactants and mixtures thereof;
wherein the amount of anionic surfactant (1) is equal to or in excess of the amount of co-surfactant (2); and wherein, if the anionic is acyl isethionate, it does not comprise more than 5% by wt. of the formulation;
(3) optionally, an alkanolamide and/or alkylamineoxide (e.g., as lather booster), wherein weight ratio of (3):(1) plus (2) is less than 1:5; (i.e., there is at least 5 times as much total surfactant as lather booster);
(4) a phase stability enhancing compound selected from the group consisting of straight chain fatty acids, CₙCOOH; where n≦14; straight chain fatty alcohols CₙCOH, wherein n≦14; branched chain fatty acids or alcohols, and unsaturated fatty acids, wherein the melting point of the branched or unsaturated fatty acid or branched alcohol is less than 45° C.; and mixtures thereof, and wherein weight ratio of (4):(1) plus (2) is 1:20 to 1:3 (e.g., from 5% to 33% of total surfactant);

(5) a lipid or lipid mimic, e.g. a lipid selected from C₁₆ and higher straight chain fatty acid, sterol, fatty ester and fatty amide; C₁₆ and higher straight chain fatty alcohols; and mixtures thereof,

wherein the selected lipid or lipid mimic has limited solubility, e.g. less than 0.5% by weight of said lipid or mimic in a surfactant solution comprising 5% by wt. of (1) plus (2); and

wherein the weight ratio of said lipid or mimic to (1) plus (2) is equal to or greater than 0.5,
wherein the composition has paste or cream like viscosity or rheology, wherein said rheology is defined by a dynamic viscosity of greater than 50 Pa·s at 1 sec⁻1, as defined in the protocol, more preferably greater than 100 Pa·s, most preferably ≧140 Pa·s, the upper limit being 1000 Pa·s (i.e., compositions do have paste or cream-like viscosity in contrast, for example, to bars, which are solid);
wherein the compositions are stable, wherein said stability is defined by absence of visual phase separation both at 25° C. and at 45° C. for 2 weeks.

The storage and loss moduli in shear experiments, as defined in protocol (i.e., as G′ and G″), is typically such that the ratio of G′ to G″ is greater than 2, preferably 3 or greater in combination with having G′ above 500 Pa, preferably above 700 Pa, more preferably equal to or above 800 Pa, measured at 1 sec⁻¹ at 25° C. In addition, the composition as noted will have paste or cream-like viscosity (in contrast, for example, to bars, which are solid), and the upper boundary for G′ is 5000 Pa.

Preferably the compositions have pH of 8 or below, preferably 5 to 8.

In addition, compositions of the invention have lather volume ≧10 milliliters (ml), e.g., 10 to 25 ml as measured by rotating cylinder lather test as defined in the protocol. The fact that lather is adequate (e.g., consumer acceptable) and comparable to that of the compositions without the same ratio of lipid to surfactant is surprising in that the lipid might be expected to severely depress lather. Thus, applicants are unpredictably able to obtain paste-like rheology for compositions which retain adequate foam.

Finally, as compositions may have for example oil continuous phase, the paste or cream like viscosity is not necessarily a function of water level. However, in a preferred embodiment, water is present in amounts greater than 25% by wt., preferably greater than 30% by wt., and even more preferably greater than 40% by wt.

### BRIEF DESCRIPTION OF THE FIGURES

FIG. 1 is an optical microscopic image of Example 7 in Table 4 of the examples observed through polarized light. The insoluble, suspended lipid material in this example is crystals of palmitic/stearic acid, which are bright when observed through cross polars. The abundance of palmitic/stearic crystals in the image reflects the limited solubility (as defined) of lipid in the surfactant system.

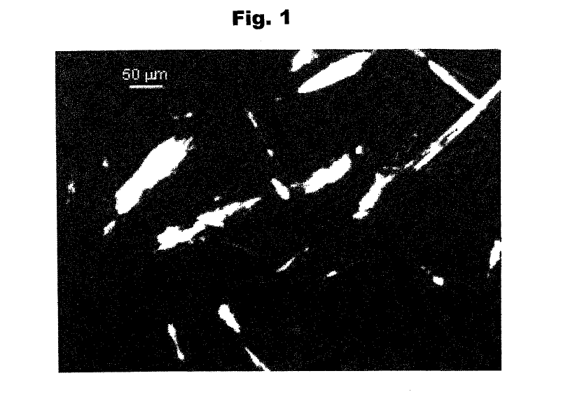

### DETAILED DESCRIPTION OF THE INVENTION

The present invention provides cleanser compositions, particularly having cream or paste-like consistency (rheological behaviour and viscosity), acceptable lather, good stability, and providing a moisturization skin feel.

Unexpectedly, applicants have found that use of critical amounts (ratios of >1:2 in relation to total surfactant) of sparingly soluble, long chain skin lipids or skin lipid mimics, particularly straight, long chain fatty acids and/or alcohols, provide excellent rheology (measured by viscosity, storage modulus and dynamic modulus, as described in the protocol) and associated moisturization while maintaining good foam and stability. By “sparingly soluble” is meant that the lipid or lipid mimics have solubility of less than 0.5% by wt. of the lipid in a surfactant solution comprising 5% by wt. of the total anionic and co-surfactant of the composition. Specifically, using the proper formulations, these long chain fatty acids and/or alcohols provide a “creamy” feel on skin (e.g., through their paste-like viscosity), and mildness, while retaining good foaming. This is accomplished, as noted, by mixing at least certain amounts (floor levels relative to total surfactant level) of sparingly-soluble, long chain lipids or lipid mimics with some amount of phase stability enhancer, for example, shorter chain length fatty acid and/or alcohol. The short chain fatty acids and/or alcohols may help provide, for example, stabilizing lamellar phase. When combined with optional use of alkanolamide and/or alkylamineoxide (foam booster), these phase enhancing shorter chain acids/alcohols may further help ensure there is good foaming in a stable composition while, simultaneously, cream-feel, rheology and moisturization are provided by the presence of longer chain fatty acids and alcohols.

In short, applicants have found a way to combine the rheological and moisture benefits provided by use of long chain fatty acids/alcohols while retaining good foam and stability despite the fact that longer chain fatty acids/alcohols would be predicted to de-foam. While not wishing to be bound by theory, it is believed the shorter chain length molecules used as phase enhancers unexpectedly serve the dual purpose of enhancing foam that would be expected to be depressed when long chain lipids are forming the “pasty” viscosity of the inventive composition. To formulate such high lipid levels relative to surfactant while retaining adequate foam is quite unpredictable.

The invention is described in more detail below.

### Primary Surfactants

The primary surfactant used in the compositions of the subject invention is anionic surfactant or surfactants, wherein the anionic surfactant has Krafft point below 20° C.

The Krafft temperature is the minimum temperature at which surfactants form micelles. A commonly accepted practical definition and one adapted here is the temperature at which a surfactant solution at 1% active exhibits no precipitated phase. Below the Krafft temperature, there is no value for the critical micelle concentration (CMC), i.e., surfactant micelles will not form. A portion of the surfactant exists as a precipitate and the mixture of surfactant and water will appear cloudy or have an obvious phase settled at the bottom of a sample below the Krafft temperature. We consider only surfactants or mixtures of surfactants with Krafft temperatures at or below 20° C., so it suffices to verify that the surfactant mixtures of interest are completely soluble at 20° C. We verified this for the commercial surfactant solutions of interest. In particular, we have verified that the Krafft temperature for mixtures of sodium lauryl-1-ether sulfate and cocoamidopropyl betaine, and for mixtures of the commercial blend known as Miracare SLB lies below 20° C. Table 1 shows the Krafft temperature for several common surfactants commonly used in personal cleansing products.

The defined Krafft point includes typical anionic surfactants such as sodium dodecyl sulfate (Krafft temperature of 18.4° C.), but would exclude, for example, surfactants such as acyl isethionate which have much higher Krafft temperature. This is an important point because, especially when large amounts of long chain fatty acids and alcohols are used (e.g., to provide “creamy” rheology and moisture feel), it is important that the Krafft temperature of this primary surfactant be relatively low.

The anionic will typically comprise 2% to 50%, preferably 5 to 40% by wt. of the composition.

### Co-Surfactant

In addition to primary anionic surfactant(s), the compositions of the invention further comprise a co-surfactant selected from the group consisting of anionic, nonionic, amphoteric, and zwitterionic surfactant and mixtures thereof.

The amount of primary anionic is in excess of the co-surfactant and, if the co-surfactant is an anionic surfactant with Krafft temperature above 20° C. (e.g. acyl isethionate), it comprises no more than 5% by wt. of the total composition.

Suitable anionic surfactants include, for example, alkyl sulfates, alkyl ether sulfates, alkali metal or ammonium salts of alkyl sulfates, alkali metal or ammonia salts of alkyl ether sulfates, alkyl phosphates, alkyl glyceryl sulfonates, alkyl sulfosuccinates, alkyl taurates, acyl taurates, alkyl sarcosinates, acyl sarcosinates, sulfoacetates, alkyl phosphate esters, mono alkyl succinates, monoalkyl maleates, sulphoacetates, acyl isethionates, alkyl carboxylates, phosphate esters, and combinations thereof.

Suitable amphoteric surfactants include, for example, betaines, alkylamido betaines, sulphobetaines, N-alkyl betaines, sultaines, amphoacetates, diamphoacetates, imidazoline carboxylates, sarcosinates, acyl amphoglycinates, such as cocamphocarboxy glycinates and acyl amphopropionates, and combinations thereof.

Zwitterionic surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is:

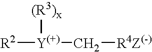

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxy alkylene of from about 1 to about 4 carbon atoms and Z is a radical selected from the group consisting of carboxylate, sulfonate, sulfate, phosphonate, and phosphate groups.

Examples of such surfactants include:

4-[N,N-di(2-hydroxyethyl)-N-octadecylammonio]-butane-1-carboxylate;

5-[S-3-hydroxypropyl-S-hexadecylsulfonio]-3-hydroxypentane-1-sulfate;

3-[P,P-diethyl-P-3,6,9-trioxatetradexocylphosphonio]-2-hydroxypropane-1-phosphate;

3-[N,N-dipropyl-N-3-dodecoxy-2-hydroxypropylammonio]-propane-1-phosphonate;

Amphoacetates and diamphoacetates are also intended to be covered as possible zwitterionic and/or amphoteric compounds which may be used.

The surfactant system may also optionally comprise a nonionic surfactant.

The nonionic which may be used includes in particular the reaction products of compounds having a hydrophobic group and a reactive hydrogen atom, for example aliphatic alcohols, acids, amides or alkyl phenols with alkylene oxides, especially ethylene oxide either alone or with propylene oxide. Specific nonionic detergent compounds are alkyl (C₆-C₂₂) phenols-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides.

The nonionic may also be a sugar amide, such as a polysaccharide amide. Specifically, the surfactant may be one of the lactobionamides described in U.S. Pat. No. 5,389,279 to Au et al. which is hereby incorporated by reference or it may be one of the sugar amides described in U.S. Pat. No. 5,009,814 to Kelkenberg, hereby incorporated into the subject application by reference.

Other surfactants which may be used are described in U.S. Pat. No. 3,723,325 to Parran Jr. and alkyl polysaccharide nonionic surfactants as disclosed in U.S. Pat. No. 4,565,647 to Llenado, both of which are also incorporated into the subject application by reference.

Preferred alkyl polysaccharides are alkylpolyglycosides of the formula

R²O(CₙH₂ₙO)ₜ(glycosyl)ₓ

wherein R² is selected from the group consisting of alkyl, alkylphenyl, hydroxyalkyl, hydroxyalkylphenyl, and mixtures thereof in which alkyl groups contain from about 10 to about 18, preferably from about 12 to about 14, carbon atoms; n is 0 to 3, preferably 2; t is from 0 to about 10, preferably 0; and x is from 1.3 to about 10, preferably from 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position.

Alkanolamide and/or Alkylamine Oxide

A third component which may optionally be used in the composition of the invention is alkanolamide, alkylamineoxide, or mixtures thereof.

The formulations of the invention may comprise 0 to 4 wt. %, preferably 0.1-4%, more preferably 0.5 to 3 wt. % of such compounds, wherein the ratio of the alkanolamide and/or alkylaminioxide to total surfactant (i.e., both primary anionic and co-surfactant) is less than 1:5 (i.e., comprises no more than 20% of total surfactants).

Examples of compounds which may be used include but are not limited to alkanolamides such as mono- and di-ethanolamides, N-methyl-monoethanolamide, isopropanolamides of fatty acids having about 10 to 20 carbon atoms, and PPG-hydroxyethyl cocamides and alkylamineoxide with carbon chain length in the range of 10 to 20. Specific examples of suitable compounds include cocomonoethanolamide, cocodiethanolamide, lauryl mono/or di ethanol amide, coco mono/or di isopropanolamide, lauryl mono/or di ethanolamide, myristyl mono/or di ethanolamide, cocoyl N-methyl-monoethanolamide, cocoylamineoxide, laurylamineoxide, myristylamineoxide, and polypropylene glycol-2-hydroxyethyl cocoamide. Particularly useful ingredients for this invention are coco mono or diethanol amide, lauryl mono/or di ethanol amide, lauryl amine oxide and coco amine oxide.

In general, these components help to maintain foam of the composition, notwithstanding the presence of large amounts of de-foaming, long chain fatty acids and alcohols. It thus further helps the longer chain lipid or lipid mimics fatty acids/alcohols to provide creaminess, as well as mildness, without the strong de-foaming that would have been expected.

If used, ratio of alkanolamide or amineoxide to total surfactant should be less than 1:5 (at higher levels, mildness may be compromised) and may be present at a level of 0.5 to 4%, more preferably 1 to 3% by wt. of the composition.

### Phase Stability Enhancers

The formulations of the invention further must comprise phase stability enhancing compounds selected from one of the following groups: (1) straight chain fatty acids, CₙCOOH, where n≦14; (2) straight chain fatty alcohols CₙCOH, wherein n≦14; (3) fatty acids or alcohols or unsaturated fatty acids, wherein the melting point of the fatty acid or alcohol is less than 45° C.; (4) fatty acid ester derivatives, and (5) mixtures thereof, and wherein weight ratio of this phase stability enhancer to anionic plus co-surfactants is 1:10 to 1:2 (i.e., the phase stabilizer comprises 10 to 50% of stabilizer plus surfactants).

In preferred embodiments, the ratio of enhancer to total surfactant is 0.1 to 0.4, preferably 0.1 to 0.3.

The compounds are important for promoting stability by modifying the phase behaviour of the surfactant phase. For example, when these compounds are included in a formulation, it is more likely that a lamellar phase, particularly a lamellar vesicle phase will be formed. A lamellar surfactant phase, if formed, provides enhanced stability with regard to phase separation and suspension of solids in the composition.

Some examples of phase stability enhancing compounds include straight chain C₈-C₁₄ fatty acid such as lauric acid and myristic acid; straight chain C₈ to C₁₄ fatty alcohols such as lauryl alcohol, and myristyl alcohol; branched chain C₈-C₂₄ fatty acid, such as isotearic acid; unsaturated C₈-C₂₄ fatty acid such as oleic; ester derivatives like propylene glycol isostearate, propylene glycol oleate, glyceryl isostearate, glyceryl oleate and similar ester derivatives. Most preferred examples include lauric acid, oleic acid, isostearic acid, and lauryl alcohol.

In addition, without wishing to be bound by theory, the phase stability enhancer appears to have dual function of ensuring adequate foam, even in the presence of lipid or lipid mimic compounds which, while applicants have found necessary to form desirable, paste-like rheology, would be expected to depress foam. Thus, the combination of phase stability enhancer and lipid or lipid mimic quite unpredictably provide a pasty product which retains adequate foaming (e.g., >7 ml, preferably ≧10 to 25 ml, measured by rotating cylinder lather test defined in protocol).

### Lipids or Skin Lipid Mimics

Compositions of the invention further comprise, as noted, lipid or skin lipid mimic compounds. Lipid may be selected from C₁₆ and higher (e.g., C₁₆-C₂₄) straight chain fatty acid, fatty esters, or fatty amides, and also may include sterols, lanosterols, other sterol derivatives, and triglycerides. In general, lipids will include any compound which satisfy the range (>1:2 ratio of lipid to total surfactant) and solubility requirements of the invention (solubility of less than 5% in a 5% solution of surfactant and cosurfactant at 25° C.). A preferred lipid composition comprises mixtures of C₁₆ and C ₁₈ fatty acid (e.g., mixture of palmitic and stearic acids). Skin lipid mimics include C₁₆ and higher straight chain fatty alcohols and mixtures thereof, and sterols and ceramides. Lipid mimics are defined to mean compounds that have hydrophobic moieties like lipids and behave like lipids in the composition with regard to solubility; they have minimal solubility in 5 wt. % surfactant solutions and insoluble crystals can be observed in prototypes when they are present. A long chain (C₁₆ and higher) unsaturated alcohol is an example of a lipid mimic, since its hydrophobic portion is identical to long-chain fatty acids, it is only sparingly soluble in 5 wt. % surfactant solutions, and solid insoluble matter will be visible in prototypes containing greater than a floor level of the material.

These lipid and lipid mimics are the key to the invention. Due to their low solubility (see below), when used in critical amounts (e.g., weight ratios relative to surfactant), i.e. greater than ½ of the level of surfactants (including cosurfactants), they provide rheological benefits (creamy feel and viscosity) as well as moisturization benefits for the skin. Further, unpredictably, in combination with phase stability enhancer, adequate foaming is maintained.

The selected lipids or lipid mimics have, as noted, limited solubility in the compositions. It is difficult to characterize the amount of insoluble lipid or lipid mimic in the inventive prototypes, or in personal wash liquids, creams, lotions, foams, pastes, or other products because of the complex phase behavior of these type of products. Optical microscopy with magnifications of 100× to 400× allow one to clearly see insoluble crystals, but the complex phase behavior, including characteristic liquid crystal patterns, make optical microscopy ineffective for quantifying the level of insoluble matter. Other methods like nuclear magnetic resonance, which measures the diffusion of hydrogens, or differential scanning calorimetry, which characterizes the heat of phase transformations, can be used to infer the ratio of solid to non-solid material, but these are indirect methods that cannot determine the level of precipitated lipid in complex surfactant mixtures and washing solutions without considerable uncertainty.

Criticality concerning the level of precipitated lipids in the inventive compositions may be defined in several ways. The lipids can be seen by optical microscopy. Thus precipitated lipid is present in the inventive compositions at room temperature (˜20 to 25° C.), as seen in micrographs. Further criticality is defined in terms of the solubility in dilute (5% by weight) solutions of the main surfactants in the compositions of the invention, since it is relatively straightforward to quantify solubility in these systems by either direct observation of solution clarity or turbidity, or by a spectrophotometric measurement. Specifically, limited solubility is defined to mean solubility of less than 0.5% by weight of said lipid or lipid mimic in a surfactant solution comprising 5% by wt. of surfactant (1) plus co-surfactants (2). The weight ratio of said lipids in the composition to surfactant (1) plus co-surfactant (2) is equal to or greater than 0.5.

The lipid solubility is quantified in a 5% by wt. surfactant as the limiting weight percent of the lipid such that the solution remains clear at temperature of 20 to 25° C. (room temperature). The solubility is a function of pH, and increases with increasing pH, but the long chain fatty acids and other lipids (e.g., cholesterol and related sterols and ceramides) and lipid mimics (e.g., saturated long chain alcohols) still have limited solubility in the surfactant solutions, especially in the pH range of interest, pH less than or equal to 8. Solubility can be determined by visual observation of a series of solutions that were allowed to equilibrate at room temperature for approximately 48 hours. We also determined solubility by absorbance of light at a particular wavelength, where we have used a UV-Vis 96 spectrophotometer well plate reader (Spectra Max, 340 pc Model) with the wavelength set at 500 nm. Results for a number of systems of interest are shown in Table 2.

In some embodiments of the invention, the lipid and/or lipid mimic may comprise 0.5 to 25% by wt., preferably 1 to 15% by wt., more preferably 3-12% by wt. of the composition. The key, however, is that there is sufficient insoluble material to form the type of turbidity characteristic of pasty compositions of the invention. Shorter chain fatty acids and more soluble lipids, by contrast, do not result in prototypes with desired cream or paste-like consistency. This is seen in the examples.

Unexpectedly, applicants have found that with inclusion of lipids at critical ratios (relative to total surfactants), the longer chain, lipid or lipid mimic compounds in the inventive cleansing composition provides a beneficial “creamy” or paste-like consistency, a moisturizing skin feel, all while the composition quite unpredictably exhibits adequate foamability (longer chain compounds would be expected to kill foaming) and phase stability. The rheological advantages noted are seen in the examples.

The formulations of the invention have a cream or paste-like viscosity defined, for example, by upper boundaries of dynamic viscosity of 1000 Pa·s and upper boundary of G′ of 5000 Pa which help distinguish over, for example, solid bar compositions. Although said cream or paste-like viscosities may be achieved in, for example, oil continuous systems, in preferred embodiments, the compositions of the invention comprise at least 30% by wt., preferably at least 40% water. Preferably, water ranges from 30% to 85% by wt.

As indicated above, the compositions have creamy or paste-like consistency. Consistency refers to rheology defined by a dynamic viscosity of greater than 50 Pa·s at 1 sec⁻¹, 25° C., as defined in the protocol, more preferably greater than 100 Pa·s, more preferably greater than 140 Pa·s and an upper boundary, as noted of 1000 Pa·s.

The compositions are stable, wherein said stability is defined by absence of visual phase separation at both 25° C. and 45° C. for 2 weeks.

Rheology may also be defined by storage and loss moduli (defined by ratio of G′ and G″). Typically, G′ is greater than 500 Pa, preferably greater than 800 Pa measured at 1 sec⁻¹ and 25° C., and upper boundary of G′ is 5000 Pa.

Preferably the compositions have pH of 8 or below.

Unpredictably as noted, the compositions also exhibit adequate foamability. Foamability can be assessed in a qualitative or quantitative way. A qualitative assessment involves a visual assessment of adequate lather in a hand wash test conducted by a person of ordinary skill. A quantitative assessment involves a test by rotating cylinder method as defined in the protocol. Typically, measured quantitatively (using rotation cylinder lather test noted in protocol below, for example), lather value ≧10 ml up to range of about 25 ml or greater depending on size of cylinder used.
Protocols
Rotating Cylinder Lather Test
Equipment
Three, 25 ml graduated cylinders with stoppers. An auto rotator, calibrated setting at 50 revolutions per minute, which allows cylinders to be securely clamped between the platforms.

### Protocol:

1) Make a 1:4 dilution of the personal wash liquid in water. This should be very well mixed/dispersed so that a homogeneous sample is used for the lather test.
2) Place 5 mls of the 1:4 dilution of the personal wash liquid sample to be tested into each of the 3 graduated cylinders.
3) Place cylinders between the platforms of the auto rotator and clamp securely.
4) Rotate samples for 50 revolutions (1 min). Allow to stand upright and drain for 30 seconds.
5) Record lather volume in Milliliters measuring lather at the top of the foam in the cylinder.
6) Lather is then reported as an average of at least three samples.
Good lather quality is defined as any value above 10 mls.

### Rheological Characterization

The prototypes were evaluated using a TA Instruments ARG2 rheometer with a cone and plate geometry. A 40 mm cone with an angle of 2 degrees 0 minutes and 42 secs (TA part number 513406.905) was used. The experimental protocol selected was that of oscillatory shear with a maximum strain of 0.5% where frequencies from 0.01 to 100 sec⁻¹ were sampled. We desire thick cleansing creams or pastes that maintained a rich creamy consistency and did not spread under gravity after being spread by the hand. It was found that the prototypes with this desired characteristic typically exhibited a viscosity at or above 100 Pascal-sec (Pa·s) when evaluated at 25° C. at a frequency of 1 sec⁻¹. The viscosity reported in an oscillatory shear experiment is understood to be the absolute value of the complex shear viscosity. The storage and loss moduli were reported for the oscillatory shear experiments, and these are commonly denoted G′ and G″, and defined by:

$$\begin{matrix}{G}^{′} = \frac{{τ}_{0}}{{γ}_{0}} cos δ \\ {G}^{″} = \frac{{τ}_{0}}{{γ}_{0}} sin δ\end{matrix}$$

where γ₀ is the magnitude of the strain imposed in the experiment, τ₀ is the magnitude of the stress measured in the experiment, and δ is the phase shift or lag between the oscillatory strain and the stress response. If the stress response is purely elastic, then there is no phase shift, δ=0, and G′=τ₀/γ₀ and G″=0. If the stress response is purely viscous, then it is completely out of phase with the imposed strain with δ=π/2 radians, and G″=τ₀/γ₀ and G′=0.

Desired prototypes typically possessed a G′ above 500 Pa, preferably above 700 Pa, more preferably above 800 Pa and a ratio of G′:G″ of approximately 3 or greater. We report G′, G″, and the viscosity in Tables 3 and 4. In some instances, as in the case of Comparative A, the system is not viscoelastic and the oscillatory shear experiment does not provide meaningful data. In such a case, we report the shear viscosity at 1 sec⁻¹ measured in a Coutte geometry. In cases where the oscillatory and steady shear experiments operate within the limits of the rheometer and the stress generated in the material is a linear function of strain and shear rate, the viscosity from the oscillatory experiment at a given frequency (1/sec) is equal to the viscosity from the steady shear at a given shear rate (1/sec). Thus, the steady shear viscosity is a valid substitute when the oscillatory shear experiment is no longer valid.

### Storage Stability

Samples were placed in 10 ml graduated cylinders, sealed, and placed in an oven at 45° C. and other samples were placed at room temperature (20° to 25° C.). Visual observations were recorded every week. All samples have some opacity initially with most having a creamy white appearance. Physical separation refers to any obvious layer formation in the sample. Different layers are discernable by disparities in opacity. Usually a small clear layer appears at the bottom of a sample.

### Solubility

Stock solutions of lipid in 5% surfactant solution were mixed in a beaker with an overhead mixer at a temperature above the melting point of the lipid for about an hour. The surfactant solution pH was adjusted to the desired range (usually 6 to 6.6) prior to addition of the lipid. Generally, the lipid concentrations in the stock solutions were at 1% to 2% by weight of solution. The stock solutions were allowed to cool to room temperature. These stock solutions were well above the solubility limit of the lipid as evidenced by considerable cloudiness. The stock solution was gently stirred to achieve a uniform suspension of the lipid insoluble matter, and then added to several small vials (approximately 20 ml capacity) in predetermined amounts. The vials were then filled with surfactant solution to create a series of dilutions of the original stock solution, which covered a range of lipid concentrations from 10⁻⁴ to 10⁻² weight % of lipid in 5 weight % surfactant solution. These samples were gently heated at 45° C. for several hours to as much as 12 hours, and then allowed to equilibrate to room temperature. The samples were observed over the course of several weeks to as long as two months to approximate the solubility limit of the lipid in the solution. Results for this visual observation of solubility are noted as Method A in Table 2. In some cases, we have determined solubility by analyzing a dilution series of samples using a UV-Vis 96 spectrophotometer (Spectra Max, Model 340pc). The samples of the dilution series were pipeted into a well plate reader and inserted into the spectrophotometer, which measured absorbance at a wavelength of 500 nm. The solubility limit was determined by extrapolating the absorbance readings to the baseline. These values are reported in Table 2 as determined by Method B.

### EXAMPLES

### Definition of Components

SLES1EO—Sodium lauryl 1-ether sulfate (surfactant) from the commercial product Steol® CS-170, Stepan
Miracare SLB—Commercial blend of sodium tridecethsulfate (surfactant), sodium lauroamphoacetate (surfactant), and cocomonoethanolamide, e.g., Miracare® SLB 365/G, from Rhodia
TDS—Sodium tridecethsulfate (surfactant)
CMEA—Cocomonoethanolamide (foam booster)
CAPBet—Cocoamidoproypyl betaine
LA—Lauric acid (lamellar phase formation)
LAA—Sodium lauroamphoacetate (surfactant)
P/SA—Commercial blend of palmitic and stearic acid, e.g., Pristerine 4911®, from Cognis
SH—Commercial blend of lanolin alcohol (about 35%), cholesterol and other sterols, e.g. Super Hartolen LD048050® from Croda.

TABLE 1
Surfactant and Surfactant Mixture Krafft Points

| Surfactant | Krafft T(° C.) |
|---|---|
| Miracare SLB | <10 |
| SLES1EO/CAPBet (5:2) | <10 |
| Trideceth Sulfate (TDS) | <15 |
| TDS/LAA | <15 |

Our operational definition of Krafft point here is the lowest temperature at which cloudiness is observed in a 1% by weight solution of surfactant. The table indicates the upper bound on Krafft point, i.e., solution observed to be clear at stated temperature.

In all cases, surfactant and/or surfactant system of the invention have Krafft temperature of 20° C. or lower and specifically exclude, for example, acyl isethionate.

TABLE 2
Solubility of Lipids in 5% Surfactant Solutions

| | | | Solubility | |
|---|---|---|---|---|
| | pH | Lipid | Limit* | Method^{#} |
| Surfactant Solution | | | | |
| SLES1EO/CAPBet 5:2 | 6.6 | P/SA | 2.5 × 10⁻³ | B |
| SLES1EO/CAPBet 5:2 | 6.0 | Stearic Acid | 1.5 × 10⁻³ | B |
| Miracare SLB | 6.0 | P/SA | 10⁻³ | A |
| Miracare SLB | 8.9 | P/SA | 7 × 10⁻³ | A |
| Miracare SLB | 6.0 | Cholesterol | ~5 × 10⁻³ | B |
| Comparative: | | | | |
| SLES1EO/CAPBet 5:2 | 6.0 | Lauric acid | ~8 × 10⁻² | A |

*Solubility limit is expressed as weight fraction of total solution.
^{#}Method: A, visual observation; B, absorbance at 500 nm, as described in text.

It is seen in all cases that solubility is less than 0.5 wt. % when measured in 5 wt. % solution of the surfactants. This is critical for creating paste-like creamy viscosity of the invention. When solubility is above 0.5 wt. %, paste-like viscosity is not observed (see comparative).

### Comparative A-F and Examples 1-11

Below are examples of the invention. Table 3 relates to sterol and betaine surfactant system and Table 4 to Miracare surfactant system.

### Tables 3 and 4

TABLE 3

| Component | Comp A | Comp B | Comp C | Ex 1 | Ex 2 | Ex. 3 | Ex 4 | Ex 5 | Ex 6 | Comp D |
|---|---|---|---|---|---|---|---|---|---|---|
| Steol CS 170 (surfactant) | 10.0 | 10.0 | 10.0 | 10.0 | 10.0 | 10.0 | 10.0 | 10.0 | 10.0 | 10.0 |
| Cocomidopropyl Betaine | 4.0 | 4.0 | 4.0 | 4.0 | 4.0 | 4.0 | 4.0 | 4.0 | 4.0 | 4.0 |
| (surfactant) | | | | | | | | | | |
| Cocoamide MEA (booster) | 2.0 | 2.0 | 2.0 | 2.0 | 2.0 | 2.0 | 2.0 | 2.0 | 0.0 | 2.0 |
| Lauric acid (phase inducer) | 3.0 | — | 3.0 | 2.0 | 2.0 | 2.0 | 2.0 | 2.0 | 3.5 | 2.7 |
| P/SA (lipid) | — | 5.0 | 5.0 | 10.0 | 10.0 | 10.0 | 10.0 | 10.0 | 10.0 | |
| Jaguar C13S (cationic) | | | | | | | | | | 0.4 |
| Petrolatum | | | | | | | | | | 10.0 |
| Glycerin | | | | 4.0 | | 20.0 | | | | 5.0 |
| Soybean oil | | | | | 4.0 | | | | | 10.0 |
| Fragrance | | | | | | 1.0 | 1.0 | 1.0 | 1.0 | 1.0 |
| NaCl | | | | | | | | 2.0 | | |
| Citric Acid | | 2.0 | | | | | | | | 0.01 |
| Water | 81.0 | 77.0 | 76.0 | 68.0 | 68.0 | 51.0 | 71.0 | 69.0 | 71.5 | 54.9 |
| Lather and stability data | | | | | | | | | | |
| Lather (milliliters) | 17.0 | 7.3 | 16.7 | 13.7 | 18.0 | 18.3 | 18.2 | 17.3 | 11.7 | 20.5 |
| Physical stability 45 C. | F | P | P | P | P | P | P | P | P | P |
| Rheology data | | | | | | | | | | |
| G′ at 1 sec−1 | | 5.4 | 234.8 | 855 | 955 | 1044 | 962 | 931 | 1059 | 138 |
| G″ at 1 sec−1 | | 13.7 | 51.6 | 199 | 249 | 70.2 | 265 | 76.9 | 143 | 48.8 |
| Viscosity at 1 sec−1 | 0.059 | 2.3 | 38.3 | 140 | 157 | 167 | 159 | 149 | 170 | 23.2 |
| Ratios of component groups | | | | | | | | | | |
| Alkanolamide: surfactants | 0.14 | 0.14 | 0.14 | 0.14 | 0.14 | 0.14 | 0.14 | 0.14 | 0 | 0.14 |
| Stability enhancer: | 0.21 | 0.0 | 0.21 | 0.14 | 0.14 | 0.14 | 0.14 | 0.14 | 0.25 | 0.19 |
| surfactants | | | | | | | | | | |
| Lipid: surfactants | 0 | 0.36 | 0.36 | 0.71 | 0.71 | 0.71 | 0.71 | 0.71 | 0.71 | 0 |

G′ = storage modulus
G″ = loss modulus
P = Pass stability test
F = Fail stability test

TABLE 4

| Component | Comp E | Comp F | Comp G | Ex 7 | Ex 8 | Ex 9 |
|---|---|---|---|---|---|---|
| Miracare (surfactant blend) | | | | | | |
| TDS | 9.6 | 9.6 | 9.6 | 9.6 | 9.6 | 9.6 |
| LAA | 4.3 | 4.3 | 4.3 | 4.3 | 4.3 | 4.3 |
| CMEA | 2.1 | 2.1 | 2.1 | 2.1 | 2.1 | 2.1 |
| Lauric acid (phase inducer) | 3 | 3 | 3 | 3 | 3 | 2 |
| P/SA (lipid) | | 5 | 5 | 10 | 10 | 10 |
| Super Hartolan (SH) | | | 5 | | | |
| Jaguar C13S | | | 0.7 | 0.7 | 0.7 | 0.7 |
| Glycerin | 1.0 | 1.0 | 1.0 | 1.0 | 30.0 | 1.0 |
| Soybean oil | | | | | 10 | |
| Citric acid | 0.4 | 0.4 | 0.4 | 0.4 | 0.4 | 0.4 |
| Water | 79.6 | 74.6 | 68.9 | 68.9 | 29.9 | 69.9 |
| Lather-Quantitative | 17.7 | 14.7 | 9 | 16 | 15.0 | 15.7 |
| Physical stability 45 C. | F | P | P | P | P | P |
| G′ at 1 sec−1 | 42.3 | 220 | 1903 | 1488 | 962 | 1326 |
| G″ at 1 sec−1 | 8.7 | 20 | 440 | 248 | 196 | 148 |
| Viscosity at 1 sec−1 | 6.9 | 35 | 311 | 240 | 156 | 212 |
| Ratios of Component Groups | | | | | | |
| Alkanolamide: surfactants | 0.15 | 0.15 | 0.15 | 0.15 | 0.15 | 0.15 |
| Stability enhancer: | 0.22 | 0.22 | 0.34 | 0.22 | 0.22 | 0.14 |
| surfactants | | | | | | |
| Lipid: surfactants | 0.00 | 0.36 | 0.59 | 0.72 | 0.72 | 0.72 |

The examples set forth in Tables 3 and 4 (Comparatives A-D and 1-6 in Table 3; and Comparatives E-F and 7-9 in Table 4) were prepared by blending surfactants and alkanolamide, if present, in water and heating to 70° C., then adding fatty acids, fatty alcohols, other lipids, or lipid mimics, oils, emollients, salts, citric acid. The mixture was stirred until homogeneous in an overhead mixer at 70° C. for at least one hour, then allowed to cool. Fragrance, if present, was added when the temperature was below 40° C. An alternative method of blending involved melting the lipids and oil, if present, adding the surfactant raw material, additional water, and the following ingredients if present: salts, glycerol, citric acid, and minor ingredients. The order of addition was not found to have a significant effect on the product rheology when it was evaluated, usually a day or more after the mixture cooled to room temperature.

Table 3 provides results for the rheological behavior, physical stability, and lather of the prototypes prepared with the surfactants SL1ES and cocoamidopropyl betaine. Table 4 provides the rheological behavior, physical stability, and lather of the prototypes prepared with the commercial blend Miracare SLB. Comparatives A, D and E show the effect of the absence of lipid, which provides structure, i.e. high storage modulus (G′) relative to loss modulus (G″) at 1 sec⁻¹ and low viscosity. For A, D and E, G′ is either unmeasurable or well below 500 Pa, even where ratio of G′ to G″ is >2 (D&E). Clearly, where there is no lipid, these examples fall below the critical ratio of lipid: surfactant of 0.5 required by the subject invention. A, D and E are all pourable liquids. These Comparatives al contain lauric acid which acts as a phase modifier, which means that the molecule is incorporated or solubilized by the surfactant phase in the Example formulations. Note in Table 2 that lauric acid solubility is determined in the 5 wt. % SLES1EO/CAPBet solution to be approximately 0.08 wt. %, or approximately 16% of the surfactant weight in a 5 wt. % surfactant solution. Lauric acid is not considered a lipid for purposes of our invention, and it is noteworthy that it does not satisfy the solubility criteria defined here for lipids, i.e. less soluble than 0.5 wt. % in a 5 wt. % surfactant solution. We have found that incorporation of lipids with lower solubility provides a creamy or paste-like rheology. The higher solubility of lauric acids is consistent with the rheological characterization, which shows that lauric acid by itself does not impart creamy or paste-like rheology for surfactant solutions.

Comparatives B, C, & F are also all pourable liquids and all have viscosity below 50 Pa·s. While Comparative C is thicker than Comparative A and Comparative F is much thicker than Comparative E, both Comparatives C and F have a lower viscosity than is desirable for a cream or paste cleansing product and will pour out of their container. Comparative A shows that viscosity is very low when no long chain fatty acid (≧C₁₆) is present. Comparative B shows that, in a composition without phase stability enhancer like a short chain fatty acid but having 5% long chain fatty acid, the viscosity, although nearly 40 times higher than A, is still very low (in the range of 2 Pa·s) and the G″>G′ at 1 sec⁻¹, again indicating that prototype is a flowable liquid. Comparative C is similar to Comparative B, although 3 wt. % lauric acid is added and the viscosity increases by a factor of more than 10. The series of Examples 1 thorough 6 with 10 wt. % long chain fatty acid (i.e., lipid >5% of (1) plus (2)) show viscosities in excess of 100 Pa·s at 1 sec⁻¹ and G′>G″ indicating the Examples are thick creamy or paste-like materials.

Comparative G is a cream like product with adequate viscosity, but it shows that lather is somewhat depressed when the components that act as stability enhancers exceed about ⅓ the weight of the surfactant and cosurfactant. SuperHartolan®, as noted in the Definition of Components above, contains about 35% lanolin alcohol, a material with a melting point in the range of 38-44° C., which satisfies the description of a phase stability enhancing component. The total amount of phase stability enhancing component in Comparative G includes both the lanolin alcohol and the lauric acid and is approximately 4.8 wt. %. Experience shows that this amount of phase stability enhancers, relative to the surfactants TDS and LAA at a total of 13.9 has a detrimental effect on lather.

Examples 1 through 6 in Table 3 and 7 through 9 in Table 4 provide illustrative examples of the invention. These formulas are creamy, have adequate lather, and are stable with respect to physical separation when held at 45° C. for longer than two weeks. G′ values are above 800 Pa measured at 1 sec⁻¹, 25° C.

1. Personal care cleanser composition comprising:
(a) 2% to 40% by wt. of an anionic surfactant having a Krafft point below 20° C.;
(b) a co-surfactant selected from the group consisting of anionic, nonionic, amphoteric, zwitterionic surfactants and mixtures thereof;
wherein the amount of (a) is equal to or in excess of the amount of (b); and wherein, anionic, if acyl isethionate, does not comprise more than 5% by. wt. of the formulation;
(c) 0% to 4% by wt. of an alkanolamide and/or alkylamineoxide,
wherein weight ratio of (c):(a) plus (b) is less than 1:5;
(d) a compound selected from the group consisting of (1) straight chain fatty acids, CₙCOOH where n≦14; (2) straight chain fatty alcohols CₙCOH where n≦14; (3) branched-chain fatty acid or alcohols and unsaturated fatty acids, wherein the melting point of the branched or unsaturated fatty acid or branched alcohol is less than 45° C.; and (4) mixtures thereof
wherein the weight ratio of (d):(a) plus (b) is 1:20 to 1:3; and
(e) a lipid or lipid mimic
wherein the weight ratio of (e):(a) plus (b) is greater than or equal to ½ and wherein lipid or lipid mimic has solubility less than 0.5% by wt. of such lipid or lipid mimic in a surfactant solution comprising 5% by wt. of (1) plus (2);
wherein composition has paste or cream like viscosity or rheology such that the viscosity is from greater than 50 Pa·s to 1000 Pa·s when measured at a frequency of 1 sec⁻¹ at 25° C.;
wherein the composition is stable, showing no signs of visual separation after storage for 2 weeks both at 25° C. and at 45° C.;
and wherein composition has lather volume ≧10 ml as measured by rotating cylinder lather test.

2. A composition according to claim 1 comprising 5 to 30% by wt. anionic surfactant.

3. A composition according to claim 1 where acyl isethionate comprise ≦1% by wt. of the composition.

4. A composition according to claim 1 wherein alkanolamide and/or amine oxide comprise 0.5-4% of the composition.

5. A composition according to claim 1 wherein phase stabilizer is lauric or myristic acid.

6. A composition according to claim 1 wherein lipid is selected from the group consisting of C₁₆-C₂₄ straight chain fatty acids, fatty esters, fatty amides; sterols; triglycerides and mixtures thereof.

7. A lipid mimic compound (having solubility of less than 0.5% in surfactant solution comprising 5% total surfactant) according to claim 1 selected from the group consisting of straight chain fatty alcohols, ceramides and mixtures thereof.

8. A composition according to claim 6 wherein lipid is mixture C₁₆-C₂₀ fatty acids.

9. A composition according to claim 1 wherein viscosity is ≧100 Pa·s to 1000 Pa·s, measured at frequency of 1 sec⁻¹ at 25° C.

10. A composition according to claim 9 wherein viscosity is ≧140 Pa·s to 1000 Pa·s, measured at frequency of 1 sec⁻¹ at 25° C.

11. A composition according to claim 1 wherein lather volume, measured by rotating cylinder lather test is ≧10 to 25 milliliters.

12. A composition according to claim 1 wherein G′ is >500 Pa to 5000 Pa measured at 1 sec⁻¹, 25° C.

13. A composition according to claim 12 wherein G′ is ≧800 Pa to 5000 Pa.

14. A composition according to claim 12, wherein the ratio of G′ to G″ is >2.

15. A composition according to claim 1 comprising 25 to 85% water.
